(19) United States
(12) Patent Application Publication
(10) Pub. No.: US 2025/0194897 A1
(43) Pub. Date: Jun. 19, 2025

(54) MEDICAL DEVICES, SYSTEMS, AND METHODS FOR DETERMINING DIMENSIONS OF A TARGET

(71) Applicant: Boston Scientific Scimed, Inc., Maple Grove, MN (US)

(72) Inventors: Longquan CHEN, Lexington, MA (US); Niraj Prasad RAUNIYAR, Plymouth, MN (US)

(73) Assignee: Boston Scientific Scimed, Inc., Maple Grove, MN (US)

(21) Appl. No.: 18/980,108

(22) Filed: Dec. 13, 2024

Related U.S. Application Data
(60) Provisional application No. 63/610,455, filed on Dec. 15, 2023.

(51) Int. Cl. A61B 1/00; A61B 1/05; A61B 1/307; G06T 7/00; G06T 7/10; G06T 7/60

(52) U.S. Cl. CPC A61B 1/000094; A61B 1/00043; A61B 1/00066; A61B 1/00165; A61B 1/05; A61B 1/307; G06T 7/0012; G06T 7/10; G06T 7/60; A61B 2576/02; G06T 2207/10016; G06T 2207/10068; G06T 2207/30084

(57) ABSTRACT

A medical system may include a medical device, a processor, and an instrument. The medical device may have a handle and a shaft defining a working channel. The shaft may have a distal opening at a distal end of the shaft. The distal end may include an imager. The instrument may be movable along the working channel to contact a target distal to the distal end of the shaft. The processor may be configured to determine a depth of the target based on pixel characteristics of the target and the instrument in at least one image generated by the imager, and based on a proximity of the instrument to the target in the at least one image.

### CROSS-REFERENCE TO RELATED APPLICATIONS

This application claims the benefit of priority to U.S. Provisional Application No. 63/610,455, filed on Dec. 15, 2023, which is incorporated by reference herein in its entirety.

### TECHNICAL FIELD

Aspects of the present disclosure generally relate to medical devices, system, and methods related thereto for determining features of a target in a body lumen. In particular, some aspects relate to medical systems, devices, and methods for determining dimensions of an object within a subject. In some aspects, the dimensions may be used to generate a three-dimensional image of the object.

### BACKGROUND

The manner in which urinary or kidney stones may be removed from patients may depend on the size of the stones. For example, some smaller stones, or fragments of stones, may be of an adequate size to pass through a bodily lumen, e.g., the urinary tract, and out of the body. In some instances, larger stones or residual fragments may be fragmented into smaller pieces, e.g., via lithotripsy, prior to removal. Some stones may require follow-ups and/or additional interventions, for example, at a later date. Size of the object can be a consideration in determining a method of removal. Size estimations are often inaccurate, owing to the inherent challenges associated with measuring a three-dimensional object from a two-dimensional image, especially when the image is of low resolution or visibility.

### SUMMARY

The present disclosure includes medical devices, systems, and related methods useful for analyzing targets such as kidney stones and/or other objects within the body. For example, the present disclosure includes a medical system that includes a medical device, a processor, and an instrument. The medical device may have a handle and a shaft defining a working channel. The shaft may have a distal opening at a distal end of the shaft. The distal end may include an imager. The instrument may be movable along the working channel to contact a target distal to the distal end of the shaft. The processor may be configured to determine a depth of the target based on pixel characteristics of the target and the instrument in at least one image generated by the imager, and based on a proximity of the instrument to the target in the at least one image.

Any of the medical systems described above and elsewhere herein may include any of the following features. The handle of the medical device may include the processor. The instrument may have a known diameter. The processor may be configured to determine the depth of the target further based on the known diameter of the instrument and contact between the instrument and the target. The processor may be configured to determine the depth of the target based on a comparison of the pixel characteristics of the instrument to the pixel characteristics of the target in the at least one image. The distal end of the instrument may abut the target in the at least one image. The instrument may include a fiber. The instrument may include a laser fiber configured to fragment the target. The at least one image may include a plurality of images. In each image of the plurality of images, the target may have a different orientation relative to the instrument.

The medical system may further include a display. The display may be configured to show the depth of the target determined by the processor with the at least one image. The processor may further be configured to perform segmentation on the at least one image.

In aspects, the processor may be further configured to determine a length and/or a width of the target based on the pixel characteristics of the target and the instrument in the at least one image and based on the proximity of the instrument to the target in the at least one image. The processor may be configured to generate a three-dimensional representation of the target. The processor may store a known diameter of the instrument and/or may be configured to prompt a user to input a known diameter of the instrument.

The present disclosure also includes methods of using the medical system described above and/or elsewhere herein for determining the depth of the target. The target may be a kidney stone. Use of the medical system may include generating a three-dimensional image of the target. The processor may determine the depth of the target based on the pixel characteristics of the target and the pixel characteristics of the instrument in a plurality of images.

The present disclosure also includes a method for analyzing a target in a body of a subject. The method may include introducing a shaft of a medical device into a lumen of the body that includes the target; positioning a distal end of the shaft proximate the target, the distal end comprising an imager; extending an instrument through a working channel of the medical device to a position proximate the target; generating, via the imager, a plurality of images of the target and the instrument; and determining, via a processor, a depth of the target based on pixel characteristics of the target and the instrument in the plurality of images.

In aspects, extending the instrument to the position may include contacting the target. The method may further comprise comparing the pixel characteristics of the target to the pixel characteristics of the instrument. Determining the depth of the target may include determining the depth of the target includes calculating a distance between the target and the instrument. The instrument may include a fiber. The target may be a kidney stone. The instrument may include a laser fiber. The method may further comprise fragmenting the target with the laser fiber.

In aspects of the present disclosure, the method may further comprise performing, via the processor, segmentation on at least one image of the plurality of images. The method may include positioning a distal end of a shaft of a medical device proximate the target, wherein the distal end of the shaft includes an imager; extending an instrument through a working channel of the medical device to a position proximate the target, wherein the instrument includes a laser fiber; generating, via the imager, at least one image of the target and the instrument; determining, via a processor of the medical device, pixel characteristics of the target and the instrument in the at least one image; and determining, via the processor, a depth of the target based on the pixel characteristics of the target and the instrument.

In aspects of the present disclosure, extending the instrument to the position may include contacting the target. The at least one image may include a plurality of images, and, in each image, the target may have a different orientation relative to the instrument. The method may further comprise rotating the target between generating a first image and generating a second image of the plurality of images.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIGS. 1A and 1B depict an exemplary medical system according to aspects of this disclosure.

FIG. 2 depicts an exemplary image obtained by a medical system, according to aspects of this disclosure.

FIG. 3 depicts a flow chart of an exemplary method, according to aspects of this disclosure.

FIGS. 4A and 4B illustrate exemplary images of a target in a first position (FIG. 4A) and in a second position (FIG. 4B), according to aspects of this disclosure.

FIG. 5 depicts a flow chart of an exemplary method, according to aspects of this disclosure.

### DETAILED DESCRIPTION

Reference will now be made in detail to aspects and examples of the present disclosure and illustrated in the accompanying drawings. Wherever possible, the same reference numbers will be used throughout the drawings to refer to the same or like parts.

Aspects of the disclosure are now described with reference to exemplary systems and methods for measuring and estimating one or more dimensions of an object in a body lumen. Some aspects are described with reference to medical procedures where a medical device is guided through a body proximate an object within a body lumen, e.g., within the urinary tract of a subject. For example, the medical device may include a shaft that can be introduced into a body lumen such as a ureter and advanced through the urethra until a distal end of the shaft is located in a calyx of a kidney, adjacent one or more kidney stones. One or more dimensions of the kidney stone may be analyzed and determined according to aspects of the present disclosure. References to a particular type of procedure, body lumen, and object are provided for illustrative purposes and not intended to limit the disclosure.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

In some techniques where a medical device with an imager is used to visualize an object in the body, such as a stone, a medical professional may seek information on the size of the object. Such information may be obtained according to the present disclosure, for example, by comparing the object to a known dimension of an instrument in an image of both the object and the instrument. Measuring or estimating the object size (including dimensions such as length, width, and/or depth) may be useful to determine an appropriate procedure for removal of the object. The width, length, and depth of an object may be used for size estimation of the object. The depth of the object refers to the dimension between a proximal face of the object to the distal face of the object. Such information may be used, for example, to generate a three-dimensional image, representation, reconstruction, or model of the object.

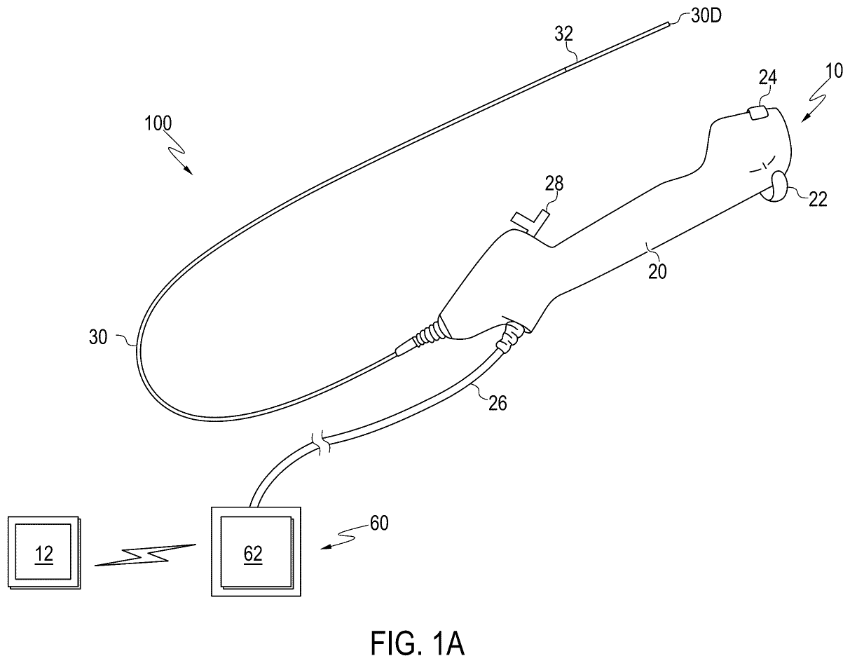

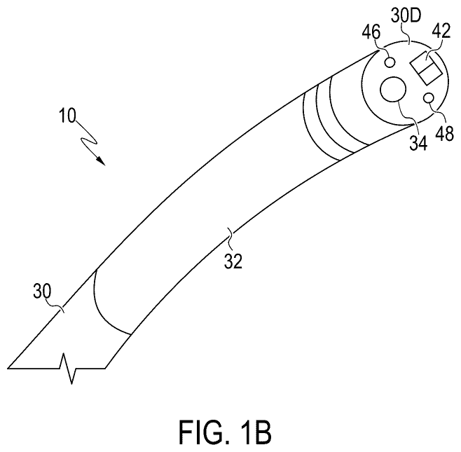

An exemplary medical system 100 useful for the present disclosure is now described with reference to FIGS. 1A and 1B. System 100 comprises a medical device 10, which may be coupled to equipment 60. For example, medical device 10 may include a ureteroscope, endoscope, bronchoscope, duodenoscope, colonoscope, etc., and may include a shaft that extends distally from a handle. Medical device 10 includes a handle 20 with at least one actuator, e.g., a first actuator 22 and a second actuator 24, a port 28, and a shaft 30 with a steerable portion 32 and distal end 30D.

Actuators 22, 24 may receive user input and transmit the user input to shaft 30. Each actuator 22, 24 may include a lever, knob, slider, joystick, button, or other suitable mechanism. For example, first actuator 22 may be configured to articulate steerable portion 32, e.g., via one or more pull wires within shaft 30, and second actuator 24 may be configured to actuate and/or control other aspects of medical device 10, e.g., turning on/off laser sources and/or capturing images.

According to some aspects of the present disclosure, equipment 60 may be configured to supply medical device 10 with vacuum/suction, fluid (e.g., liquid, air), and/or power via umbilicus 26. As shown in FIG. 1A, equipment 60 may include a processor 62, e.g., operable with medical device 10. For example, processor 62 may help generate a visual representation of image data and/or transmit the visual representation to one or more interface devices, e.g., a display 12. According to some aspects, processor 62 may augment the visual representation. Display 12 may include, e.g., a touch-screen display or other display, capable of displaying images, optionally with information regarding features shown in the image(s), generated using medical device 10 and processor 62.

While FIG. 1A shows processor 62 as part of equipment 60 coupled to medical device 10, additionally or alternatively, medical device 10 may include processor 62. For example, processor 62 may be included in handle 20 of medical device 10.

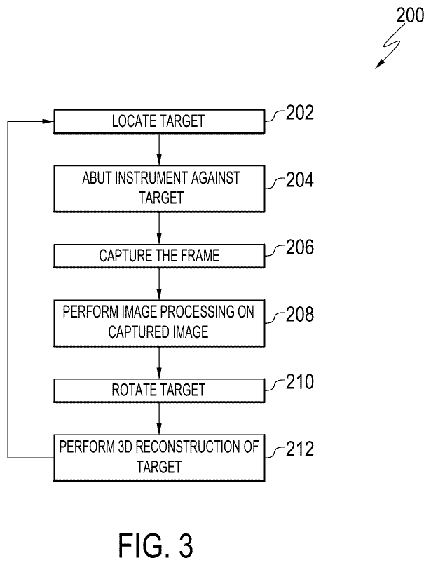

Port 28 of handle 20 may include one or more openings in communication with a working channel 34 of shaft 30. An instrument such as, e.g., a fiber 90 (e.g., laser fiber), grasper, retrieval device, etc., may be inserted through port 28 and moved distally through shaft 30 through working channel 34 to exit working channel 34 distal to shaft 30. Shaft 30 may further include one or more lumen(s) for receiving pull wires and/or other wiring, cables, and/or fluidics tubing. While the discussion herein generally refers to fiber 90 as an exemplary instrument movable along working channel 34 (or alongside medical device 10) and used in analysis of a target, it is understood that the present disclosure is not limited to a fiber. One or more alternative instruments (e.g., a guidewire, a basket, a snare, forceps, etc.) may be used in place of or in addition to fiber 90. Accordingly, aspects of fiber 90 discussed below may be applicable to any instrument used, for example, to perform one or more aspects of method 200 discussed in connection to FIG. 3.

As shown in FIG. 1B, distal end 30D of shaft 30 may include a distal opening of working channel 34, imager 42 (e.g., camera or other imaging device), and a light source 46. In some examples, shaft 30 may include a cap covering portions of distal end 30D, e.g., protecting features such as imager 42 and/or light source 46. In some examples, imager 42 may include a camera comprising a CMOS sensor. In some examples, imager 42 may include fiber optics in communication with a sensor or other device within shaft 30 or handle 20. In some examples, light source 46 may include a plastic optical fiber (POF) device or a light-emitting diode (LED). Optionally, distal end 30D may include a sensor 48, such as a pressure sensor, a distance sensor, a light sensor, a temperature sensor, an ultrasonic sensor, etc.

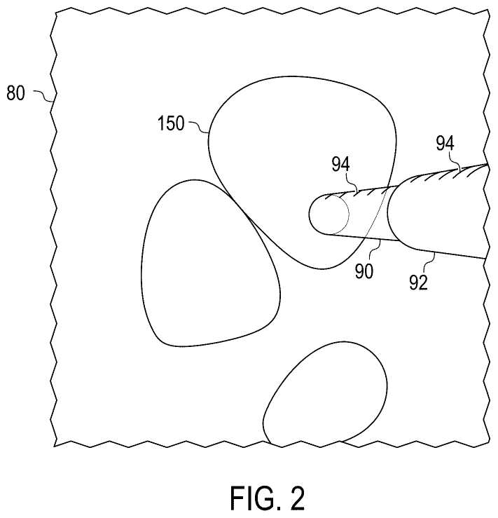

FIG. 2 depicts an exemplary image 80 generated via an imager of a medical device according to the present disclosure, such as imager 42 of medical device 10. Image 80 may be shown on display 12. In generating image 80 via processor 62, distal end 30D (and thus imager 42) of medical device 10 may be proximate a target 150. While target 150 may be various anatomical or foreign objects, in this example, target 150 is depicted as a kidney stone.

Fiber 90 may be movable within working channel 34 of shaft 30. Fiber 90 may be extended distally through working channel 34 such that at least a portion of fiber 90 exits working channel 34 to be distal to distal end 30D of medical device 10. In some aspects, fiber 90 may be insertable alongside shaft 30. In some aspects, at least a portion of fiber 90 may include a sheath 92.

Fiber 90 may include a laser fiber, e.g., capable of fragmenting a stone via laser lithotripsy, for example, or may comprise an optical fiber. Fiber 90 may comprise a transparent or translucent material, which may provide for the ability to visualize a portion of target 150 that is behind fiber 90 (e.g., otherwise obscured from view by an opaque fiber or by the sheath 92). In other aspects, fiber 90 may comprise an opaque material.

Optionally, fiber 90 and/or sheath 92 may include one or more markings 94. The one or more markings 94 may be indicative of a distance relative to distal end 30D of medical device 10. In some examples, markings 94 may be regularly spaced apart from each other (e.g., a distance of 1 mm, 1.5 mm, 2 mm, etc. between adjacent markings 94). For example, the one or more markings 94 and/or other features of fiber 90 may be used to approximate a length of fiber 90 extending distally from distal end 30D of medical device 10.

Dimensions of fiber 90 such as diameter and length may be known and used to determine dimensions of target 150, e.g., by proximity of fiber 90 to target 150 and comparison of pixel characteristics of fiber 90 and target 150 in image 80. For example, a diameter of fiber 90 may be known and stored by processor 62. In aspects, the diameter may be manually inputted into medical system 100 by a user (e.g., using a keyboard or other input device) and stored by processor 62. In some aspects, the diameter of fiber 90 may be preprogrammed into processor 62.

Additionally or alternatively, the diameter of fiber 90 may be automatically inputted into medical system 100 and stored in processor 62. For example, a machine-readable image (e.g., a barcode or quick-response code) associated with fiber 90 may be scanned via a scanning device, camera, or other reading device. Information associated with the machine-readable image may be transferred and stored by processor 62 and/or or other aspects of medical system 100. For example, the machine-readable image associated with fiber 90 may include information such as the diameter of fiber 90, length of fiber 90, type of fiber 90 (e.g., laser operating parameters if fiber 90 is a laser fiber) and/or other characteristics of fiber 90, which may be automatically inputted into processor 62 after being scanned. In these aspects, processor 62 and/or other aspects of medical system 100 may be configured to decode the machine-readable image associated with fiber 90 and/or retrieve information stored remotely, e.g., on a server, via a link or other pointer provided by the machine-readable image.

Processor 62 (or other aspects of medical system 100) may be configured to use one or more images, e.g., image 80, to determine one or more dimensions of target. For example, processor 62 may be configured to determine (e.g., estimate) a size of target 150, such as the length and/or depth of target 150. In aspects, processor 62 may be configured to perform segmentation. Segmentation, also known as contouring or annotation, is an image processing technique used to delineate regions in an image. For example, segmentation may be performed on image 80 to delineate the outer edges of target 150 (e.g., target segmentation) and/or the outer edges of fiber 90 (e.g., instrument segmentation). Segmentation may include feature detection and/or feature extraction. For example, features of target 150 may be identified via processor 62. The identified features may be matched between subsequent images. Optionally, medical system 100 may generate a three-dimensional image, e.g., via processor 62.

With both the outer edges of fiber 90/sheath 92 and target 150 and the distance of target 150 to distal end 30D known, processor 62 may be configured to calculate a number of pixels associated with each of fiber 90/sheath 92 and target 150. The number of pixels associated with target 150 may then be used to calculate one or more dimensions of target 150 (e.g., a width and/or a height). For example, because a diameter of fiber 90 is known, a relationship between pixel count and one or more dimensions of target 150 may be provided.

In some aspects, the distal end of fiber 90 may have a known diameter of approximately 1.0 mm. Depending on the distance of which fiber 90 is extended distally from distal end 30D of medical device 10, the number of pixels in image 80 displaying fiber 90 may change. As fiber 90 is extended distally from distal end 30D of medical device 10, fiber 90 may be shown in image 80 using fewer pixels. Alternatively, when fiber 90 is more proximal to distal end 30D, fiber 90 may be shown in image 80 using additional pixels. For example, the distal end of fiber 90 may be shown in image 80 using 100 pixels. Processor 62 may be configured to use this known ratio between the number of pixels associated with the distal end of fiber 90 and the known size of the distal end of fiber 90 to determine the size of target 150 when fiber 90 abuts target 150. For example, when the distal end of fiber 90 abuts target 150, a width of target 150 may be shown using 200 pixels in image 80. Accordingly, the width of target 150 may be approximately 2.0 mm. Many other dimensions (e.g., a height, a cross-sectional diameter, etc.) of target 150 may also be calculated using this known ratio.

In some aspects, the user may manually input a known distance between target 150 and distal end 30D. For example, the user may use markings 94 on fiber 90 and/or sheath 92 to ascertain the distance between target 150 and distal end 30D.

Additionally or alternatively, the image processing techniques performed by processor 62 may include using captured image 80 to calculate a distance of the distal end of fiber 90 from distal end 30D of medical device 10. Thus, processor 62 may be configured to calculate a distance between the proximal surface/face of target 150 and the distal end 30D of medical device 10. The distance to target 150 from distal end 30D may be used, for example, to further assist in generating a 3D image of target 150.

In aspects of the present disclosure, the distance to target 150 from distal end 30D may be determined according to a known width of fiber 90. For example, a width of the distal end of fiber 90 may be determined in pixels from captured image 80 (e.g., by processor unit 62). The width of the distal end of fiber 90 in pixels may be compared to a calibration table manually or automatically, e.g., via processor unit 62. Calibration may be performed to correlate the fiber width in pixels in an image and the distance between the tip of the fiber 90 and imager 42 (i.e., the distance between the tip of fiber 90 and distal end 30D of shaft 30 that includes imager 42). In some aspects, processor 62 may store calibrated parameters according to known dimensions of fiber 90, e.g., calibration performed prior to use. For example, fiber 90 may be extended from distal end 30D by a first distance. A first image (e.g., similar to image 80) may be generated. The pixel width of fiber 90 in the first image may then be associated with the first distance. Fiber 90 may then be extended by a second distance. A second image may be captured. The pixel width of fiber 90 in the second image may then be associated the second distance. Fiber 90 may be extended more or less from distal end 30D and more images may be captured to increase the number of data points used for calibration. During a procedure, processor 62 may then use the pixel width and distance data of fiber 90 to determine one or more measurements of target 150. Exemplary calibration data is provided below in Table 1.

TABLE 1

| Fiber width | Distance between fiber |
|---|---|
| (pixels) | tip and imager (mm) |
| 100 | 2 |
| 80 | 2.5 |
| 60 | 3 |
| 40 | 3.5 |

FIG. 3 illustrates an exemplary method 200 of analyzing a target in a body of a subject, e.g., target 150. Method 200 may be performed with any of the medical systems or devices discussed herein (e.g., medical system 100). For example, in step 202, distal end 30D of medical device 10 may be introduced into a body lumen of the subject and advanced to a location proximate target 150. Before, during, or after distal end 30D is proximate of target 150, an instrument, such as fiber 90, may be inserted through working channel 34 of shaft 30. In step 204, a distal end of fiber 90 may contact (abut or touch) target 150. For example, fiber 90 may be extended distally relative to distal end 30D until the distal end of fiber 90 abuts target 150. Additionally or alternatively, medical device 10 may be moved distally such that the distal end of fiber 90 abuts target 150. A medical professional or other user may confirm that fiber 90 contacts target 150 by visual confirmation (e.g., observing the distal end of fiber 90 in contact with target 150, optionally by observing movement of target 150 due to contact with fiber 90), and/or by tactile feedback.

In step 206, for example, when fiber 90 contacts target 150, one or more images (see, e.g., image 80 in FIG. 2) may be generated. In some aspects, the image(s) may be transmitted and stored in a processor of the medical system (e.g., processor 62) and/or a memory associated with any other aspect of the medical system. In some aspects, the image(s) may be generated and saved to an external memory (e.g., an external database). In step 208, the processor may process the image 80, e.g., performing segmentation of the target and/or instrument.

In step 210, target 150 may be rotated, e.g., by contact with fiber 90 and/or a fluid (e.g., a liquid or gas). For example, the medical device may be used to deliver a liquid and/or gas towards target 150 to rotate 150. Target 150 may be rotated (e.g., by less than 360 degrees less than 270 degrees, or less than 180 degrees) such that at least one feature detected during segmentation is in view. For example, target 150 may be rotated such that at least a portion of a surface of target 150 shown in a previous image is also shown in the subsequent image.

Once target 150 has been rotated to change the orientation of target 150 relative to the instrument (e.g., fiber 90) and relative to the imager of the medical device (e.g., imager 42), steps 202-210 may be repeated one or more times. For example, fiber 90 may contact target 150 (e.g., step 204) causing partial rotation of target 150, and one or more subsequent images generated (e.g., step 206). Image processing may be performed on the newly generated image, as discussed above (e.g., step 208). For example, segmentation may be performed on the newly generated image. Processor 62 may be configured to identify one or more features in each subsequent image. As steps 202-210 are repeated, more features may be detected and more measurements of target 150 may be calculated.

In a step 212, processor 62 may be configured to perform three-dimensional (3D) reconstruction of target 150. For example, the generated images and optionally associated data may be compiled to generate a 3D representation of target 150. In some aspects, the 3D representation of target 150 generated by processor 62 may be shown on display 12. Processor 62 may be configured to perform image processing discussed above on each image in real time (e.g., during a procedure). In other aspects, processor 62 may be configured to save each image and perform image processing as discussed above on each image at a later time (e.g., after the procedure). As more images are generated, a more defined 3D image of target 150 may be generated.

In generating a 3D image, representation, or reconstruction of target 150, the medical system may provide information regarding size (depth, length, and/or width), volume, and/or shape of target 150. This information may be used, for example, to determine how to remove target 150. For example, the dimensions of target 150 may be used to determine if target 150 may be removed via working channel 34 of medical device 10 or whether it may be appropriate to reduce the size of target 150 prior to removal. For example, a medical professional may perform lithotripsy or other procedure on target 150. In some aspects, the determined dimensions of target 150 and/or other information may be stored and compared to target 150 at a later point in time, for example, to determine growth, shrinkage, and/or other changes of target 150 over time.

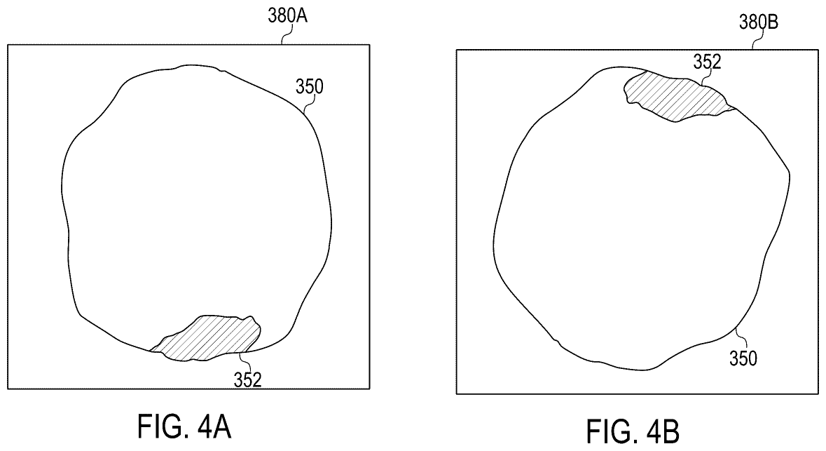

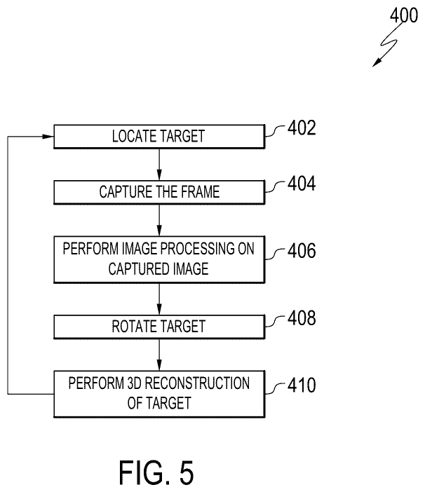

FIGS. 4A and 4B illustrate exemplary images 380A, 380B generated for another target 350 according to aspects of the present disclosure. Target 350 is depicted as a kidney stone but may be any anatomical or foreign object. FIG. 5 illustrates an alternative exemplary method 400 for analyzing target 350 using images 380A, 380B. Method 400 may be performed with any of the medical systems, devices, or portions of systems discussed herein (e.g., medical system 100). In step 402, a medical device (e.g., medical device 10) may be introduced into a body lumen of a subject to a location proximate target 350. With target 350 located and in view of an imager of the medical device, image 380A (FIG. 4A) may be generated in step 404. Image 380A may be stored to a processor (e.g., saved to a memory of processor 62). Processor 62 may be configured to determine dimensions of target 350, for example, by leveraging simultaneous localization and mapping (SLAM) techniques. In aspects, these techniques may be used to estimate a size of target 350 and/or generate a 3D model/image of target 150. For example, in step 406, processor 62 may perform image processing on image 380A. The image processing may include segmentation and/or feature extraction on target 350. For example, processor 62 may identify at least one point or region 352 of target e.g., on a proximal surface of target 350. The at least one point or region 352 may be at or near an outer edge of target 350 visible in image 380. In other aspects, the at least one point or region 352 may be near a center of target 350.

In step 408, target 350 may be rotated to change the orientation between target 350 and fiber 90 and/or between target 350 and an instrument. FIG. 4B illustrates exemplary image 380B with target 350 rotated relative to the position of target 350 shown in FIG. 4A. For example, an instrument (e.g., fiber 90 discussed above, or any other instrument) may be used to rotate target 350. Additionally or alternatively, a fluid (e.g., a liquid or gas) may be delivered (e.g., via medical device 10 or another instrument) to rotate target 350. Target 350 may be rotated in any direction (e.g., by less than 180 degrees) such that at least one point or region 352 detected during feature extraction on target 350 from image 380A is still in view.

Once target 350 is rotated, steps 402-408 may be repeated one or more times. For example, target 350 may be relocated by moving medical device 10 within the subject (e.g., step 402). An image of target 350 may be generated (e.g., step 404). Processor 62 may be configured to perform image processing on the generated image (e.g., step 406). For example, segmentation and/or feature extraction may be performed on the newly generated image. Processor 62 may be configured to identify at least one point or region 352 between each subsequent image. Target 350 may then be rotated again (e.g., step 408). As steps 402-408 are repeated, more features of target 350 may be detected.

In a final step 410, processor 62 may be configured to analyze dimensions of target and/or generate a 3D image of target 350. For example, using the data collected from the segmentation and/or feature extraction on each of the images (e.g., image 380A, 380B, etc.), a 3D image may be generated of target 350.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical system comprising:
a medical device including a handle and a shaft defining a working channel having a distal opening at a distal end of the shaft, the distal end also including an imager;
a processor; and
an instrument movable along the working channel to contact a target distal to the distal end of the shaft;
wherein the processor is configured to determine a depth of the target based on pixel characteristics of the target and the instrument in at least one image generated by the imager, and based on a proximity of the instrument to the target in the at least one image.

2. The medical system of claim 1, wherein the handle of the medical device includes the processor.

3. The medical system of claim 1, wherein the instrument has a known diameter and the processor is configured to determine the depth of the target further based on the known diameter of the instrument and contact between the instrument and the target.

4. The medical system of claim 1, wherein the processor is configured to determine the depth of the target based on a comparison of the pixel characteristics of the instrument to the pixel characteristics of the target in the at least one image.

5. The medical system of claim 1, wherein a distal end of the instrument abuts the target in the at least one image.

6. The medical system of claim 1, wherein the instrument includes a laser fiber configured to fragment the target.

7. The medical system of claim 1, wherein the at least one image includes a plurality of images, and in each image of the plurality of images, the target has a different orientation relative to the instrument.

8. The medical system of claim 1, further comprising a display configured to show the depth of the target determined by the processor with the at least one image.

9. The medical system of claim 1, wherein the processor is further configured to perform segmentation on the at least one image.

10. The medical system of claim 1, wherein the processor is further configured to determine a length and/or a width of the target based on the pixel characteristics of the target and the instrument in the at least one image and based on the proximity of the instrument to the target in the at least one image.

11. A method of analyzing a target in a body of a subject, the method comprising:
introducing a shaft of a medical device into a lumen of the body that includes the target;
positioning a distal end of the shaft proximate the target, the distal end comprising an imager;
extending an instrument through a working channel of the medical device to a position proximate the target;
generating, via the imager, a plurality of images of the target and the instrument; and
determining, via a processor, a depth of the target based on pixel characteristics of the target and the instrument in the plurality of images.

12. The method of claim 11, wherein extending the instrument to the position includes contacting the target, and the method comprises comparing the pixel characteristics of the target to the pixel characteristics of the instrument.

13. The method of claim 12, wherein determining the depth of the target includes calculating a distance between the target and the instrument.

14. The method of claim 11, wherein the instrument includes a fiber.

15. The method of claim 11, wherein the target is a kidney stone.

16. The method of claim 15, wherein the instrument includes a laser fiber and the method further comprises fragmenting the target with the laser fiber.

17. The method of claim 11, further comprising performing, via the processor, segmentation on at least one image of the plurality of images.

18. A method of analyzing a target in a body of a subject, the method comprising:
positioning a distal end of a shaft of a medical device proximate the target, wherein the distal end of the shaft includes an imager;
extending an instrument through a working channel of the medical device to a position proximate the target, wherein the instrument includes a laser fiber;
generating, via the imager, at least one image of the target and the instrument;
determining, via a processor of the medical device, pixel characteristics of the target and the instrument in the at least one image; and
determining, via the processor, a depth of the target based on the pixel characteristics of the target and the instrument.

19. The method of claim 18, wherein extending the instrument to the position includes contacting the target, and wherein the at least one image includes a plurality of images, and in each image the target has a different orientation relative to the instrument.

20. The method of claim 19, further comprising rotating the target between generating a first image and generating a second image of the plurality of images.
